(19) United States
(12) Patent Application Publication
(10) Pub. No.: US 2026/0108145 A1
(43) Pub. Date: Apr. 23, 2026

(54) SMART FIXATION DEVICE

(71) Applicant: ALCON INC., Fribourg (CH)

(72) Inventor: Steven T. Charles, Memphis, TN (US)

(21) Appl. No.: 19/353,699

(22) Filed: Oct. 9, 2025

Related U.S. Application Data
(60) Provisional application No. 63/710,107, filed on Oct. 22, 2024.

(51) Int. Cl. A61B 3/00; G06F 3/01

(52) U.S. Cl. CPC A61B 3/0091; A61B 3/0008; A61B 3/0025; G06F 3/013

(57) ABSTRACT

Embodiments of the present disclosure provide improved fixation devices and fixation targets for use during optometric or ophthalmic procedures, including diagnostic and surgical procedures.

### INTRODUCTION

In numerous ophthalmological diagnostic and/or surgical procedures, a patient is provided with a target, called a “fixation target,” to focus one or both eyes upon. Generally, the fixation target can be used to measure eye movements or help the patient maintain a stable gaze during the procedure.

Conventionally, the fixation target includes a light generated by a fixation device having a light source. The fixation device is often attached at a distal end of an articulated, multi-link mechanism that is manually held by a physician or an assistant during the procedure. Such fixation devices, however, have several limitations.

### SUMMARY

Embodiments of the present disclosure provide improved fixation devices and fixation targets for use during optometric or ophthalmic procedures, including diagnostic, laser, and surgical procedures.

In certain embodiments, a system is provided, the system comprising: an optometric or ophthalmic device for performing one or more types of biometric analyses, surgical laser procedures, or imaging procedures; a fixation device coupled to the optometric or ophthalmic device, the fixation device comprising: a near-to-eye (NTE) display device, the NTE display device configured to: generate a fixation target in response to receipt of a first electrical control signal; and at least one liquid lens, the at least one liquid lens configured to: propagate the generated fixation target to an eye of a patient; and deform in shape in response to receipt of a second electrical control signal, wherein the deformation in shape of the at least one liquid lens changes a focal plane of the propagated fixation target; and a controller in communication with the fixation device, the controller configured to: transmit the first electrical control signal to the NTE display device to cause the generation of the fixation target; and transmit the second electrical control signal to the at least one liquid lens to cause the deformation in shape of the at least one liquid lens

In certain embodiments, a fixation device is provided, the fixation device comprising: a near-to-eye (NTE) display device, the NTE display device configured to: generate a fixation target in response to receipt of a first electrical control signal; at least one liquid lens, the at least one liquid lens configured to: propagate the generated fixation target to an eye of a patient; and deform in shape in response to receipt of a second electrical control signal, wherein the deformation in shape of the at least one liquid lens changes a focal plane of the propagated fixation target; and a controller in communication with the NTE display device and the at least one liquid lens, the controller configured to: transmit the first electrical control signal to the NTE display device to cause the generation of the fixation target; and transmit the second electrical control signal to the at least one liquid lens to cause the deformation in shape of the at least one liquid lens.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 is a front view of an optical biometry system for use with the fixation devices described herein, according to certain embodiments of the present disclosure.

FIG. 2 is a perspective view of an imaging/visualization system for use with the fixation devices described herein, according to certain embodiments of the present disclosure.

FIG. 3 is a schematic plan view of a biometry and/or visualization system with a fixation device, according to certain embodiments of the present disclosure.

FIG. 4 is a schematic plan view of another biometry and/or visualization system with a fixation device, according to certain embodiments of the present disclosure.

FIG. 5 is a perspective view of a surgical laser system for use with the fixation devices described herein, according to certain embodiments of the present disclosure.

FIG. 6 is a schematic plan view of a surgical laser system integrated with a fixation device, according to certain embodiments of the present disclosure.

FIG. 7 is a schematic plan view of another surgical laser system integrated with a fixation device, according to certain embodiments of the present disclosure.

FIG. 8 is a schematic plan view of a fixation device, according to certain embodiments of the present disclosure.

FIG. 9 is a schematic plan view of a fixation device, according to certain embodiments of the present disclosure.

FIG. 10 is an exemplary fixation target generated by the fixation devices described herein, according to certain embodiments of the present disclosure.

FIG. 11 is an exemplary fixation target generated by the fixation devices described herein, according to certain embodiments of the present disclosure.

FIG. 12 is an exemplary fixation target generated by the fixation devices described herein, according to certain embodiments of the present disclosure.

FIG. 13 is an exemplary fixation target generated by the fixation devices described herein, according to certain embodiments of the present disclosure.

FIG. 14 is an exemplary fixation target generated by the fixation devices described herein, according to certain embodiments of the present disclosure.

FIG. 15 represents a patient’s field of view (FOV) during performance of eye tracking with the fixation devices described herein, according to certain embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

A fixation target can be used to measure eye movements or help a patient maintain a stable gaze during an optometric or ophthalmic diagnostic and/or surgical procedure. Conventionally, the fixation target includes a circular or dot-like light generated by a handheld fixation device having a light source. The fixation device is often mounted at a distal tip of an articulated, multi-link mechanism that is manually held by a physician or their assistant during a procedure. As such, the device and generated light are disposed only a few inches away from the patient’s eye during use. For hyperopic patients, focusing on such a closely-positioned target can prove strenuous, and can result in patient discomfort and/or difficulty in maintaining a stable gaze during the procedure. Further, the circular and/or dot-like pattern of the light generated by conventional fixation devices may be difficult to focus on, or even visualize, by patients suffering from certain retinal diseases such as age-related macular degeneration (AMD) due to defects in the patient’s central visual field. Still further, patients will often become distracted and look away from a fixation target during a procedure, and manually refocusing their gaze with conventional fixation devices can greatly impede the performance of an optometric or ophthalmic procedure or cause multiple delays, thereby resulting in frustration for the patient and physician or assistant.

Embodiments of the present disclosure provide improved fixation devices and improved fixation targets for use during optometric or ophthalmic procedures, including diagnostic and surgical procedures.

In certain embodiments, the fixation devices disclosed herein may be utilized for diagnostic procedures, including optical biometry assessments. In such embodiments, the fixation devices may be coupled with, or even integrated with, an optical biometer or other similar device.

In certain embodiments, the fixation devices disclosed herein may be utilized for ophthalmic surgical procedures, including laser-assisted cataract surgery (LACS). In such embodiments, the fixation devices may be coupled with, or even integrated into, a surgical laser system, such as a femtosecond laser-assisted cataract surgery (FLACS) system, a laser-assisted in situ keratomileusis (LASIK) system, a laser epithelial keratomileusis (LASEK) system, a photorefractive keratectomy (PRK) system, small incision lenticular extraction (SMILE) surgery system, smooth incision lenticular keratomileusis (SILK), a yttrium aluminum garnet (YAG) laser system for capsulectomy and/or iridectomy procedures, a laser system for selective laser trabeculoplasty and/or other glaucoma procedures, a laser system for retinal photocoagulation procedures, or other similar system. However, other non-laser-assisted ophthalmic surgical procedures are also contemplated, such as phacoemulsification or robotic cataract surgical procedures.

In certain embodiments, the fixation devices disclosed herein may be utilized for optometric or ophthalmic imaging and/or visualization. For example, the fixation devices may be utilized for visualization of a patient’s eye during vitreoretinal or cataract surgery. In such embodiments, the fixation devices may be coupled with, or even integrated into, a surgical imaging/visualization system or other similar system for visualization of a patient’s eye during vitreoretinal or cataract surgery. In certain embodiments, the fixation devices may be coupled with, or integrated with, other types of imaging/visualization devices.

In certain embodiments, the fixation devices disclosed herein may be utilized for controlling natural accommodation of a patient’s eye during diagnostic procedures, surgical procedures, and/or imaging/visualization procedures. As used herein, “accommodation” refers to the ability of the eye to change a refractive power of its lens to automatically focus on objects at various distances.

Examples will now be described relative to the Drawings.

Note that, as described herein, a “distal” end, side, or portion of a component refers to the end, side, or the portion that is closer to the patient’s body during the use thereof (e.g., at a far end away from the surgical device). On the other hand, a “proximal” end, side, or portion of the component refers to the end, side, or portion that is distanced further away from the patient’s body (e.g., is closer to the surgical device).

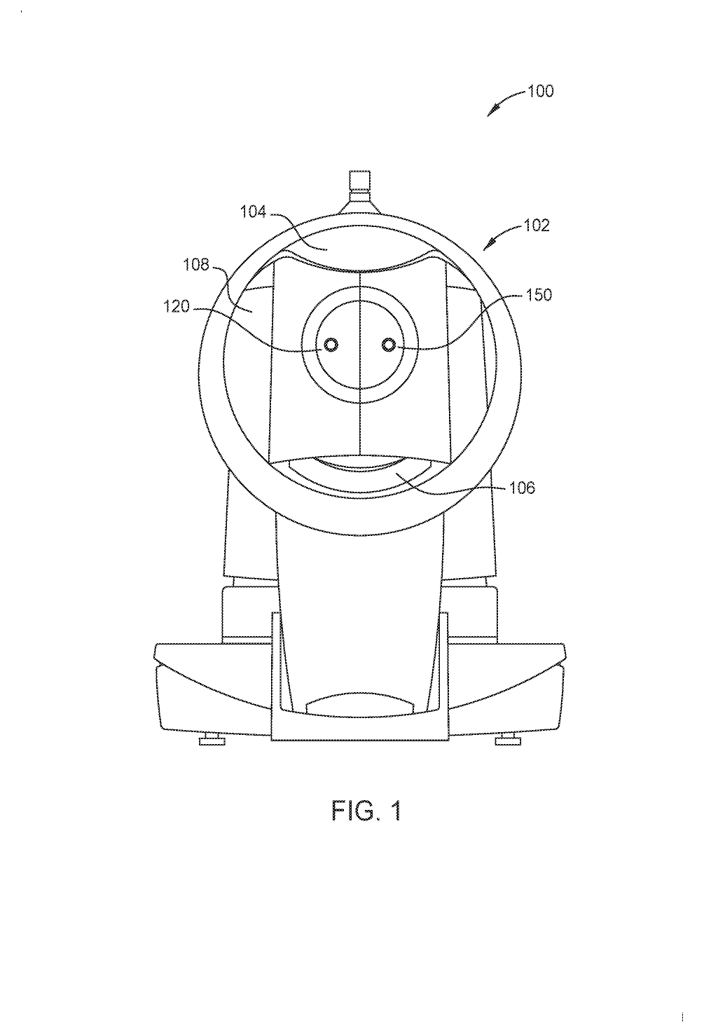

FIG. 1 is a front view of an example optometric or ophthalmic diagnostic system 100 for use with the fixation devices described herein, according to certain embodiments of the present disclosure. The diagnostic system 100 can be or include one or more optometric or ophthalmic testing devices, including without limitation, an optical biometer, an optical coherence tomography (OCT) instrument such as a swept source-OCT (SS-OCT) biometer, an OCT retina and/or anterior segment imaging system, other low-coherence interferometry instrument, a conventional or wide-angle fundus camera for taking images of the eye, a digital fundus camera, an instrument for fundus autofluorescence (AF) and/or multispectral imaging, an instrument for taking measurements of the eye for diagnosis or pre-operative planning such as for cataract or vitreoretinal surgery, an instrument for generating data about the eye, a keratometer, an autorefractor, a topography measurement device such as a corneal topography device, a device for tear film assessment, an ultrasound device such as a B-scan ultrasound device, or combinations thereof. Other types of preoperative, perioperative (i.e., interoperative), and/or postoperative testing devices are also contemplated.

In FIG. 1, the diagnostic system 100 is shown as including an upright optical biometer 102 having a headrest 104, chin rest 106, and measurement module 108; however, the description below is equally applicable to other types of optometric or ophthalmic testing devices, including those described above. The optical biometer 102 includes a first optical system 120 (for example, within the measurement module 108) that includes a succession of optical devices, such as one or more sensors, detectors, light sources, lenses, mirrors, beam splitters, beam combiners, projection systems, reflecting prisms, filters and thin films, fiber optics, and/or the like, for performing one or more diagnostic functions of the optical biometer 102. In certain embodiments, the optical biometer 102 includes, or is in communication with, one or more displays of the diagnostic system 100 configured to display a graphical user interface (GUI) for controlling one or more aspects of the diagnostic system 100.

The diagnostic system 100 further includes a fixation device 150 that is configured to generate a fixation target for a patient to focus one or both of their eyes during use of the diagnostic system 100. In certain embodiments, the fixation device 150 is integrated with the optical biometer 102 (e.g., the fixation device 150 is a component of the optical biometer 102) but is distinct from the optical system 120 such that it comprises its own optical system and/or optical devices for generating a fixation target. In certain embodiments, the fixation device 150 is integrated with the optical biometer 102 and with the optical system 120 such that it shares one or more optical devices with the optical system 120, or such that an optical pathway of the fixation device 150 overlaps with an optical pathway of the optical system 120. In certain embodiments, the fixation device 150 is adjustably and/or removably attached to the optical biometer 102. For example, the fixation device 150 may be adjustably and/or removably attached to a periphery of the optical biometer 102 (for example, to a side of the measurement module 108). Other arrangements, however, are also contemplated.

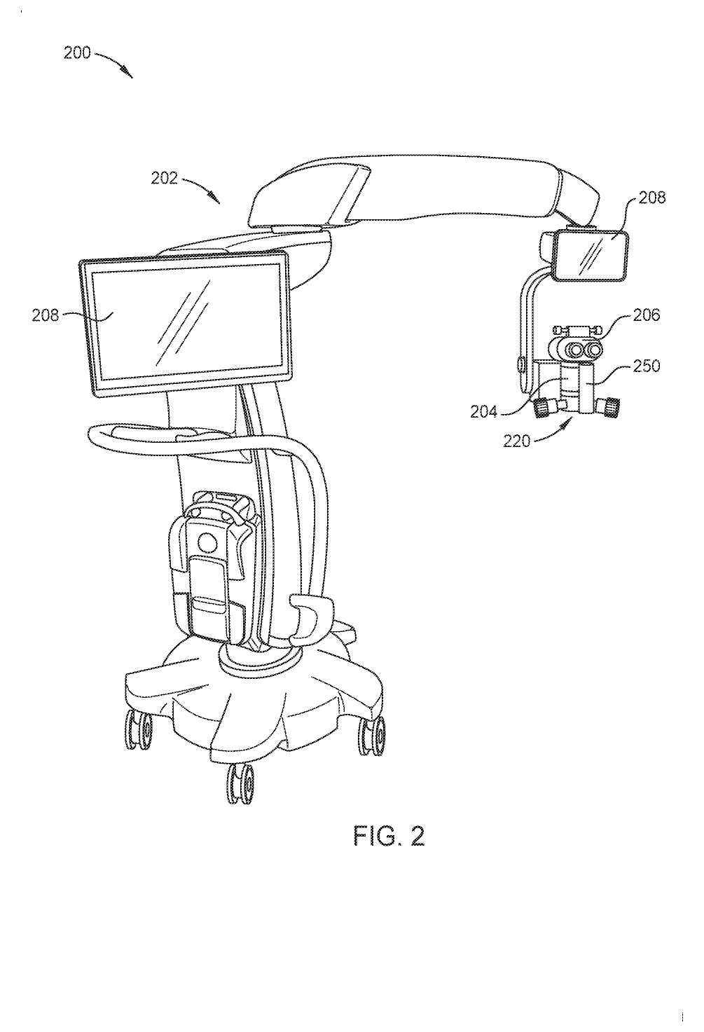

FIG. 2 is a perspective view of an example optometric or ophthalmic visualization system 200 for use with the fixation devices described herein, according to certain embodiments of the present disclosure. The visualization system 200 can be or include one or more devices for optometric or ophthalmic imaging and/or visualization, including without limitation, a microscope for magnification of the eye and its structures, a camera for taking images of the eye, an OCT system, a fundus autofluorescence system, a slitlamp biomicroscope, an ophthalmoscope, an ultrasound device, a device for visual field testing such as microperimetry, or combinations thereof. Other types of preoperative, perioperative, and/or postoperative imaging and/or visualization devices are also contemplated.

In FIG. 2, the visualization system 200 is shown as including a surgical microscope 202 including a microscope head 204, oculars 206, and one or more integrated displays 208; however, the description below is equally applicable to other types of optometric or ophthalmic imaging and/or visualization devices, including those described above. The surgical microscope 202 has a first optical system 220 (for example, within the microscope head 204) that includes a succession of optical devices, such as one or more sensors, detectors, light sources, lenses, mirrors, beam splitters, beam combiners, projection systems, reflecting prisms, dispersing devices, filters and thin films, fiber optics, and/or the like, for performing one or more functions of the exemplary optometric or ophthalmic imaging and/or visualization devices described above. In certain embodiments, the one or more displays 208 are configured to display a graphical user interface (GUI) for controlling one or more aspects of the visualization system 200.

The visualization system 200 further includes a fixation device 250 that is configured to generate a fixation target for a patient to focus one or both of their eyes during use of the visualization system 200. In certain embodiments, the fixation device 250 is integrated with the surgical microscope 202 but is distinct from the optical system 220 such that it comprises its own optical system and/or optical devices for generating a fixation target. In certain embodiments, the fixation device 250 is integrated with the surgical microscope 202 and with the optical system 220 such that it shares one or more optical devices with the optical system 220, or such that an optical pathway of the fixation device 250 overlaps with an optical pathway of the optical system 220. In certain embodiments, the fixation device 250 is adjustably and/or removably attached to the surgical microscope 202. For example, the fixation device 250 may be adjustably and/or removably attached to a periphery of the surgical microscope 202 (for example, to a side of the microscope head 204, as shown in FIG. 2). Other arrangements, however, are also contemplated.

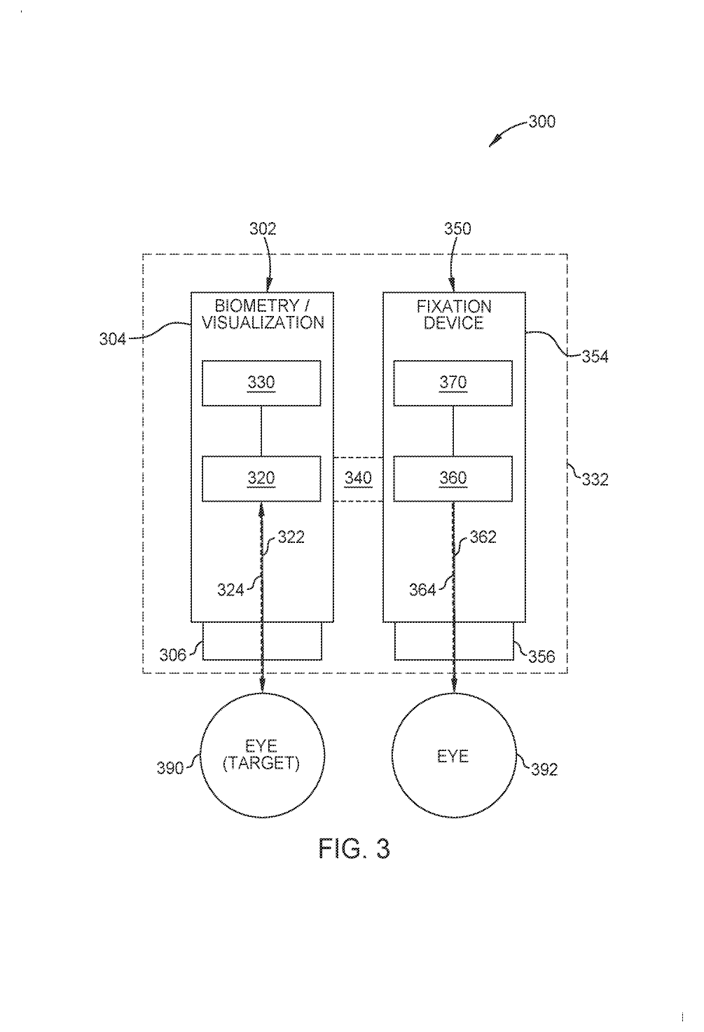

FIG. 3 is a schematic plan view of a biometry and/or visualization system 300 with a fixation device 350, according to certain embodiments of the present disclosure. Generally, the biometry and/or visualization system 300 can be representative of the diagnostic system 100 and/or the visualization system 200 described above.

As shown, the biometry and/or visualization system 300 includes a biometry and/or visualization device 302 that may include, for example, an optical biometer, OCT ophthalmoscope, or the like. The biometry and/or visualization device 302 includes its own optical system 320, which may include one or more near-to-eye (NTE) display devices (such as light-emitting diode (LED) display devices, organic light-emitting diode (OLED) display devices, micro-light emitting diode (microLED) display devices, liquid crystal display (LCD) devices, active-matrix liquid crystal display (AMLCD) devices, liquid crystal on silicon (LCOS) display devices, ferroelectric liquid crystal on silicon (fLCOS) display devices, other silicon-based display panels, other microdisplay types including those manufactured by Magin Corporation (Hopewell Junction, NY), SeeYA Technology (Hefei, China), and Kopin Corporation (Westborough, MA), and the like), sensors, detectors, light sources, lenses, mirrors, beam splitters, beam combiners, projection systems, reflecting prisms, filters and thin films, fiber optics, and/or the like for acquiring and/or analyzing data and/or images of a patient’s target eye 390. Imaging and/or visualization performed utilizing the optical system 320 may include the use of near infrared and/or visible imaging techniques. The optical system 320 is at least partially, and in certain embodiments, entirely, disposed within a housing 304 of the biometry and/or visualization device 302. The optical system 320 forms a bi-directional optical pathway 322 that extends at least partially through the housing 304 and exits the housing 304 at a patient interface 306 along an optical axis 324. In certain embodiments, the patient interface 306 includes a lens or other device of the optical system 320, such as an objective lens, for propagating light along the optical axis 324 to and/or from the target eye 390. Generally, the biometry and/or visualization device 302, the optical system 320, and/or the patient interface 306 may be arranged such that the optical axis 324 can be aligned, or arranged co-axially, with an optical or visual axis of the target eye 390 during use. In certain embodiments, the optical axis 324 is boresighted with the patient interface 306.

The optical system 320 is in communication with at least one controller 330 of the biometry and/or visualization device 302. Generally, the controller 330 may include a processor and associated memory, and can be utilized to control one or more components of the biometry and/or visualization device 302, such as the optical system 320, in performing one or more functions of the biometry and/or visualization device 302.

The fixation device 350 is coupled to the biometry and/or visualization device 302. In certain embodiments, the fixation device 350 includes its own housing 354, which may be attached to the housing 304 of the biometry and/or visualization device 302 via a coupling 340. In certain embodiments, the coupling 340 includes a fixed, or non-adjustable, coupling between the housing 354 and the housing 304. In certain embodiments, the coupling 340 includes a mechanically adjustable coupling such that a position of the housing 354 may be adjusted relative to a position of the housing 304. In certain embodiments, the coupling 340 includes a detachable coupling such that the housing 354 may be removably attached to the housing 304. In certain embodiments, the coupling 340 includes an adjustable and detachable coupling. In further embodiments, the fixation device 350 and the biometry and/or visualization device 302 are integrated into a shared housing 332. In some embodiments, the coupling 340 may include the housing 354 and the housing 304 being physically and distinctly coupled to the shared housing 332 while being in electric, data, or other communication with each other.

In FIG. 3, the fixation device 350 includes an optical system 360 separate from the optical system 320. Generally, the optical system 360 includes a light or image source and one or more lenses for generating and propagating a fixation target along an optical pathway 362 that exits the fixation device 350 through a patient interface 356 and along an optical axis 364 separate from the optical axis 324. In certain embodiments, the patient interface 356 includes a lens or other device of the optical system 360, such as an objective lens, for propagating the fixation target along the optical axis 364 to a non-target eye 392 (e.g., a contralateral eye) of a patient. In certain embodiments, the optical axis 364 is boresighted with the patient interface 356.

The fixation device 350, the optical system 360, and/or patient interface 356 may be fixedly or adjustably arranged such that the optical axis 364 is aligned, or arranged co-axially, with an optical or visual axis of the non-target eye 392 during use. In certain embodiments, the fixation device 350, and/or components thereof, and the biometry and/or visualization device 302, and/or components thereof, are arranged such that the optical axis 364 and the optical axis 324 are disposed at an interpupillary distance (PD) of a patient. For example, the optical axis 364 and the optical axis 324 are disposed between about 50 mm and about 80 mm from one another, such as between about 60 mm and about 70 mm from one another. In certain embodiments, the distance between the optical axes 364 and 324, or the distance between the fixation device 350 and the biometry and/or visualization device 302, or the distance between the patient interface 356 and the patient interface 306, can be adjusted via the coupling 340. In certain embodiments, the fixation device 350, and/or components thereof, and the biometry and/or visualization device 302, and/or components thereof, are arranged such that the optical axis 364 and the optical axis 324 are parallel or non-parallel to one another.

In certain embodiments, the optical system 360 is in communication with at least one controller 370 of the fixation device 350. Generally, the controller 370 may include a processor and associated memory, and can be utilized to control one or more components of the fixation device 350, such as the optical system 360, in performing one or more functions of the fixation device 350. In certain embodiments, the controller 370 and the controller 330 are one and the same. In other words, the fixation device 350 and the biometry and/or visualization device 302 may share a controller.

In certain embodiments, the controller 370 and/or controller 330 are in communication with a user interface of the biometry and/or visualization device 302, such as a graphical user interface (GUI) displayed on a screen of the biometry and/or visualization device 302, and from which the controller 370 and/or controller 330 may receive user inputs for controlling the fixation device 350. For example, in certain embodiments, the controller 370 and/or controller 330 may receive user inputs from the user interface for controlling a working distance (e.g., focal plane), shape, size, color, brightness, contrast, position, and/or other parameters of the fixation target generated by the fixation device 350. As a further example, the controller 370 and/or the controller 330 may receive user inputs to adjust the distance between the optical axes 324 and 364. In certain embodiments, the user interface includes a GUI, a touchpad, one or more buttons, a joystick, a foot pedal, or other user input devices. In certain embodiments, the fixation device 350 includes its own user interface for controlling the fixation device, which may include one or more of the user input devices described above.

During use of the biometry and/or visualization system 300, the patient may focus the non-target eye 392 on the fixation target generated by the fixation device 350, while light and/or images are propagated to and/or from the target eye 390 of the patient by the biometry and/or visualization device 302 for performance of diagnostic and/or visualization functions.

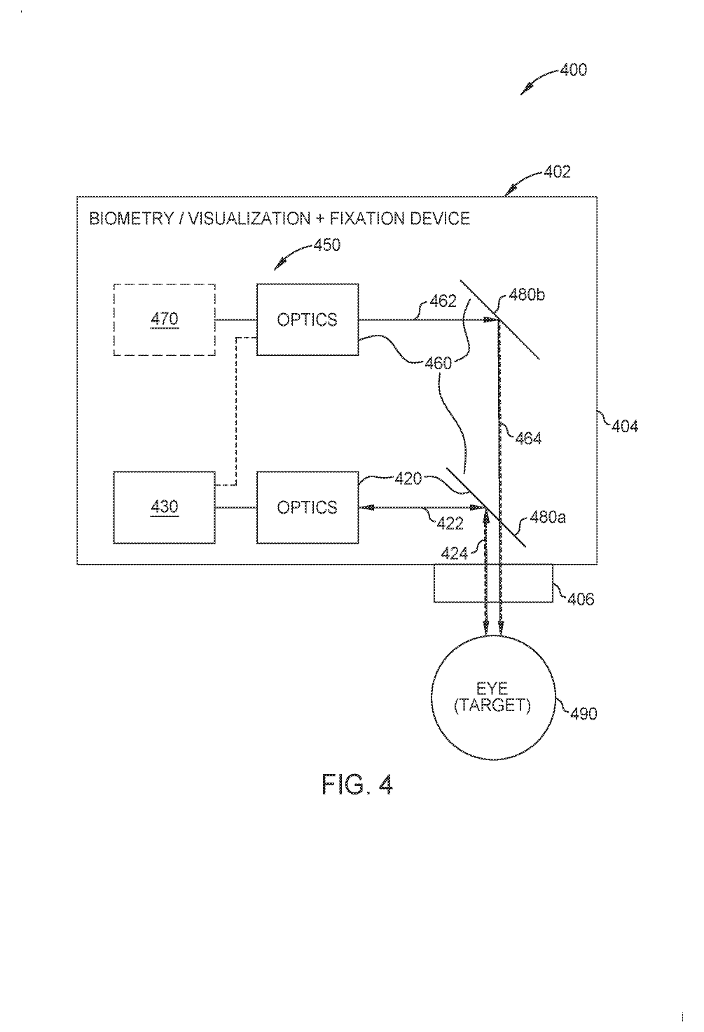

FIG. 4 is a schematic plan view of a biometry and/or visualization system 400 with a fixation device 450, according to certain embodiments of the present disclosure. Generally, the biometry and/or visualization system 400 can be representative of the diagnostic system 100 and/or the visualization system 200 described above.

As shown, the biometry and/or visualization system 400 includes a biometry and/or visualization device 402 that may include, for example, an optical biometer or OCT retinal or anterior segment imaging system. The biometry and/or visualization device 402 includes a first optical system 420 for performing one or more diagnostic functions of the biometry and/or visualization device 402. For example, the first optical system 420 may include one or more sensors, detectors, light sources, lenses, mirrors, beam splitters, beam combiners, projection systems, reflecting prisms, dispersing devices, filters and thin films, fiber optics, and/or the like, for acquiring and/or analyzing data and/or images of a patient’s eye(s). Imaging and/or visualization performed with the optical system 420 may include near infrared and/or visible imaging techniques. The optical system 420 is at least partially, and in certain embodiments, entirely, disposed within a housing 404 of the biometry and/or visualization device 402. The optical system 420 forms a bi-directional optical pathway 422 that extends at least partially through the housing 404 and exits the housing 404 at a patient interface 406 along an optical axis 424. In certain embodiments, the patient interface 406 includes a lens or other device of the optical system 420, such as an objective lens, for propagating light along the optical axis 424 to and/or from a target eye 490 of a patient. In certain embodiments, the optical axis 424 is boresighted with the patient interface 406.

The optical system 420 is in communication with at least one controller 430 of the biometry and/or visualization device 402. Generally, the controller 430 may include a processor and associated memory, and can be utilized to control one or more components of the biometry and/or visualization device 402, such as the optical system 420, in performing one or more functions of the biometry and/or visualization device 402.

The fixation device 450 is integrated with the biometry and/or visualization device 402 and is disposed within the housing 404. The fixation device 450 includes an optical system 460 that may include a light or image source and one or more lenses for generating and propagating a fixation target. In the embodiments of FIG. 4, the optical system 460 overlaps with the optical system 420. In other words, the optical system 460 and the optical system 420 share one or more optical devices (e.g., lenses, mirrors, beam splitters, beam combiners, etc.), and/or form one or more coaxial optical axes. For example, as shown in FIG. 4, the optical system 460 propagates the fixation target along an optical pathway 462 that exits the biometry and/or visualization device 402 through the patient interface 406 along an optical axis 464 that is coaxial with the optical axis 424.

To facilitate coaxial alignment of the optical axis 464 and the optical axis 424, the optical system 460 and/or optical system 420 may include one or more optical devices 480 configured to redirect and/or combine light generated and propagated by the optical system 460 and/or optical system 420. Such optical devices 480 may include mirrors, beam splitters, beam combiners, and/or the like. In FIG. 4, the optical system 460 includes an optical device 480b, which may be a mirror (such as a dichroic mirror), while both the optical system 460 and the optical system 420 share an optical device 480a, which may be a beam splitter or beam combiner. This arrangement facilitates the overlapping of the optical axis 424 of the optical system 420 and the optical axis 464 of the optical system 460, which then both pass through the patient interface 406. Generally, the biometry and/or visualization device 402, the optical system 420, the optical system 460, and/or the patient interface 406 may be arranged such that the optical axis 424 and optical axis 464 can be aligned, or arranged co-axially, with an optical or visual axis of the target eye 490 during use. In certain embodiments, the optical axes 424a, 424b, and/or 464 are boresighted with the patient interface 406.

In certain embodiments, the optical system 460 is in communication with at least one controller 470 of the fixation device 450. Generally, the controller 470 may include a processor and associated memory, and can be utilized to control one or more components of the fixation device 450, such as the optical system 460, in performing one or more functions for generating and propagating a fixation target. In certain embodiments, however, the fixation device 450 (and/or optical system 460) shares a controller with the optical system 420 and/or other components of the biometry and/or visualization device 402.

In certain embodiments, the controller 470 and/or controller 430 are in communication with a user interface of the biometry and/or visualization device 302, such as a graphical user interface (GUI) displayed on a screen of the biometry and/or visualization device 402, and from which the controller 470 and/or controller 430 may receive user inputs for controlling the fixation device 450. For example, in certain embodiments, the controller 470 and/or controller 430 may receive user inputs from the user interface for controlling a working distance (e.g., focal plane), shape, size, color, brightness, contrast, position, and/or other parameters of the fixation target generated by the fixation device 450. In certain embodiments, the user interface includes a GUI, a touchpad, one or more buttons, a joystick, a foot pedal, or other user input devices.

During use of the biometry and/or visualization system 400, the patient may focus the target eye 490 on the fixation target generated by the optical system 460, while light and/or images are simultaneously propagated to and/or from the target eye 490 by the optical system 420 for performance of diagnostic and/or visualization functions of the biometry and/or visualization device 402.

In certain embodiments, additional optical devices such as mirrors (e.g., dichroic mirrors), beam splitters, beam combiners, and the like may be used to split the optical pathway 462 into two optical pathways to direct the fixation target out of the biometry and/or visualization device 402 along two optical axes (including optical axis 464). For example, the optical pathway 462 may be split into two pathways, where one pathway passes through the patient interface 406 and the other pathway passes through an additional patient interface (not shown in FIG. 4). This facilitates the propagation of the fixation target to both the target eye 490 and a non-target eye of the patient. In such embodiments, this arrangement may be used to form a three-dimensional fixation target, which may help the patient to maintain better focus as compared to a two-dimensional fixation target propagated to only one eye of the patient.

FIG. 5 is a perspective view of a surgical laser system 500 for use with the fixation devices described herein, according to certain embodiments of the present disclosure The surgical laser system 500 can be or include one or more devices for performing ophthalmic surgical laser procedures, including without limitation, a LACS system, a FLACS system, a laser-assisted in situ keratomileusis (LASIK) system, a laser epithelial keratomileusis (LASEK) system, a photorefractive keratectomy (PRK) system, small incision lenticular extraction (SMILE) surgery system, smooth incision lenticular keratomileusis (SILK), a yttrium aluminum garnet (YAG) laser system for capsulectomy and/or iridectomy procedures, a laser system for selective laser trabeculoplasty (SLT) and/or other glaucoma procedures, a laser system for retinal photocoagulation procedures, a laser system for phacoemulsification, a laser system for vitreolysis such as a femtosecond laser for vitreolysis, a robotic laser system, components thereof, or combinations thereof. Other types of perioperative surgical devices are also contemplated. In FIG. 5, the surgical laser system 500 is shown as a FLACS system 502 including a single-piece patient attachment interface 504 (i.e., an eye docking device), a laser optical head 506, a laser system chassis 508, and on or more displays 510; however, the description below is equally applicable to other types of ophthalmic surgical laser devices, including those described above. In certain embodiments, the one or more displays 510 are configured to display a graphical user interface (GUI) for controlling one or more aspects of the surgical laser system 500.

The FLACS system 502 has a first optical system 520 (for example, within the laser optical head 506 and the patient attachment interface 504) that includes a succession of optical devices, such as one or more sensors, detectors, light sources, lenses, mirrors, projection systems, reflecting prisms, dispersing devices, filters and thin films, fiber optics, and/or the like, for performing one or more functions of the exemplary ophthalmic surgical laser devices described above. The FLACS system 502 further includes a fixation device 550 that is configured to generate a fixation target for a patient to focus one or both of their eyes upon during use of the FLACS system 502.

In certain embodiments, the fixation device 550 is fixedly integrated with the FLACS system 502 but distinct from the optical system 520 such that it comprises its own optical system and/or optical devices for generating a fixation target. In certain embodiments, the fixation device 550 is fixedly integrated with the FLACS system 502 and with the optical system 520 such that it shares one or more optical devices with the optical system 520, or such that an optical relay pathway of the fixation device 550 overlaps with an optical relay pathway of the optical system 520. In certain embodiments, the fixation device 550 is adjustably and/or removably attached to the FLACS system 502. For example, the fixation device 550 may be adjustably and/or removably attached to a periphery of the FLACS system 502 (for example, to a side of the patient attachment interface 504, as shown in FIG. 5). Other arrangements, however, are also contemplated.

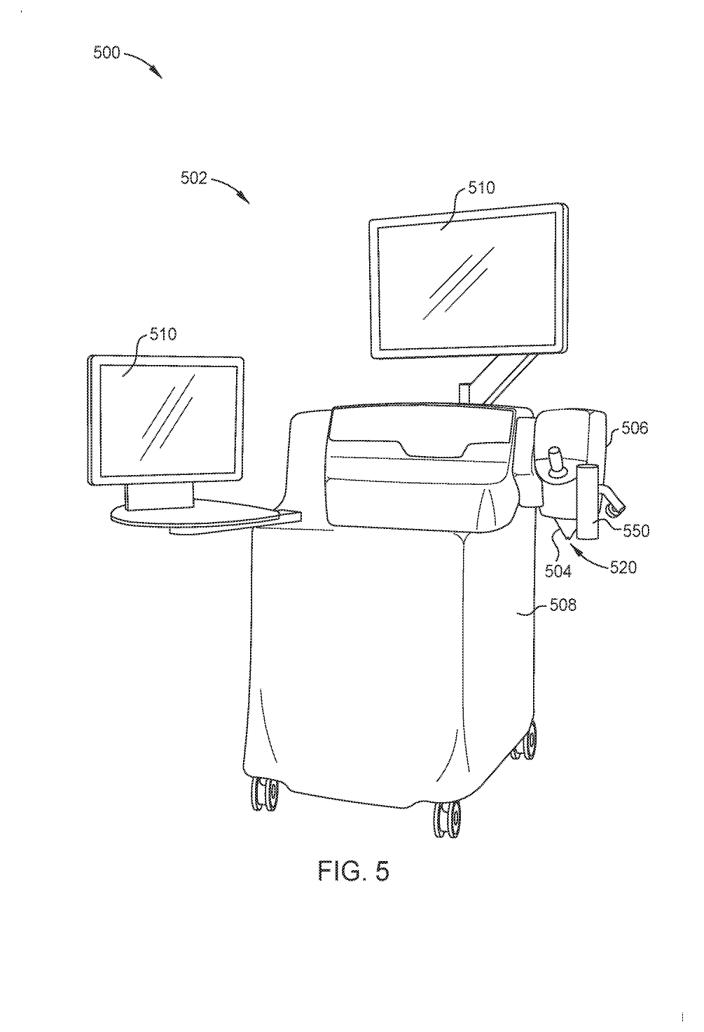

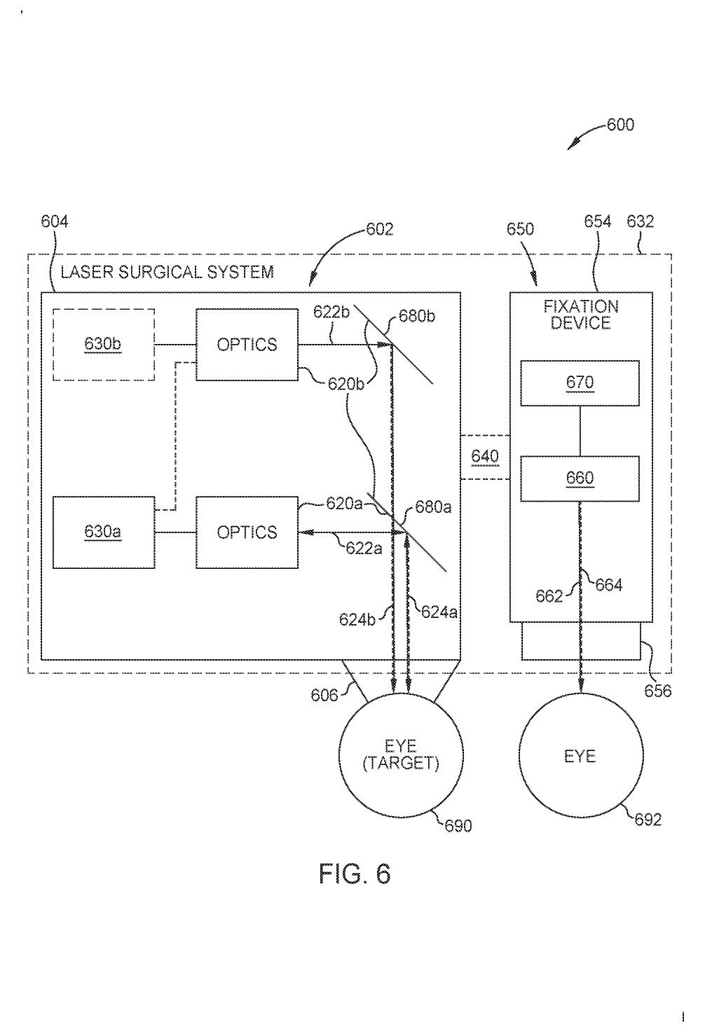

FIG. 6 is a schematic plan view of a surgical laser system 600 with a fixation device 650, according to certain embodiments of the present disclosure. Generally, the surgical laser system 600 can be representative of the surgical laser system 500 described above.

As shown, the surgical laser system 600 includes a surgical laser device 602 that may include, for example, a FLACS device or system. The surgical laser device 602 includes at least one optical system 620a and/or 620b, which may include one or more sensors, detectors, light sources, laser sources, lenses, mirrors, beam splitters, beam combiners, projection systems, reflecting prisms, dispersing devices, filters and thin films, fiber optics, and/or the like for performing one or more functions of the surgical laser device 602. Example functions include imaging/visualization and laser treatment. In certain embodiments, the surgical laser device 602 includes at least two optical systems 620a and 620b, wherein each of the at least two optical systems 620a and 620b performs different functions of the surgical laser device 602. The optical system 620a and/or the optical system 620b are at least partially, and in certain embodiments, entirely, disposed within a housing 604 of the surgical laser device 602.

In FIG. 6, the optical system 620a propagates light for imaging/visualization functions along a bi-directional optical pathway 622a that extends at least partially through the housing 604 and exits the housing 604 at a patient interface 606 along an optical axis 624a. Imaging and/or visualization performed with the optical system 620a may include near infrared and/or visible imaging techniques. Meanwhile, the optical system 620b propagates laser light for laser treatment functions along an optical pathway 622b that extends at least partially through the housing 604 and exits the housing 604 at the patient interface 606 along an optical axis 624b. In certain embodiments, the patient interface 606 includes a lens or other device, such as an objective lens, for propagating light along the optical axis 624a and/or optical axis 624b to and/or from a target eye 690 of a patient. Generally, the surgical laser device 602, the optical system 620a, the optical system 620b, and/or the patient interface 606 may be arranged such that the optical axis 624a and/or the optical axis 624b can be aligned, or arranged co-axially, with an optical or visual axis of the target eye 690 during use. In certain embodiments, the optical axis 624 is boresighted with the patient interface 606.

In certain embodiments, the at least two optical systems 620a and 620b can overlap in the surgical laser device 602. In other words, the optical system 620a and the optical system 620b can share one or more optical devices (e.g., lenses, mirrors, beam splitters, beam combiners, etc.), and/or form one or more coaxial optical axes. For example, in the example of FIG. 6, the optical axis 624a and optical axis 624b overlap (e.g., are coaxial) for at least portions thereof. To facilitate coaxial alignment of the optical axis 624a and the optical axis 624b, the optical system 620a and/or optical system 620b may include one or more optical devices 680 configured to redirect and/or combine light generated and propagated by the optical system 620a and/or optical system 620b. Such optical devices 680 may include mirrors, beam splitters, beam combiners, and/or the like. In FIG. 6, the optical system 620b includes an optical device 680b, which may be a mirror (such as a dichroic mirror), while both the optical system 620a and the optical system 620b share an optical device 680a, which may be a beam splitter or beam combiner. This arrangement facilitates the overlapping of the optical axis 624a of the optical system 620a and the optical axis 624b of the optical system 620b, which then both pass through the patient interface 606.

The optical system 620a and/or 620b is in communication with at least one controller 630 of the surgical laser device 602. Generally, the controller 630 may include a processor and associated memory, and can be utilized to control one or more components of the surgical laser device 602, such as the optical system 620a and/or 620b, in performing one or more functions of the surgical laser device 602. In certain embodiments, each of the optical system 620a and the optical system 620b are in communication with a corresponding and separate controller 630 (e.g., illustrated as controllers 630a and 630b, respectively, either or both of which may be referred to as controller 630).

The fixation device 650 is coupled to the surgical laser device 602. In certain embodiments, the fixation device 650 includes its own housing 654, which may be attached to the housing 604 of the surgical laser device 602 via a coupling 640. In certain embodiments, the coupling 640 includes a fixed, or non-adjustable, coupling between the housing 654 and the housing 604. In certain embodiments, the coupling 640 includes a mechanically adjustable coupling such that a position of the housing 654 may be adjusted relative to a position of the housing 604. In certain embodiments, the coupling 640 includes a detachable coupling such that the housing 654 may be removably attached to the housing 604. In certain embodiments, the coupling 640 includes an adjustable and detachable coupling. In further embodiments, the fixation device 650 and the surgical laser device 602 are integrated into a shared housing 632. In some embodiments, the coupling 640 may include the housing 654 and the housing 604 being physically and distinctly coupled to the shared housing 632 while being in electric, data, or other communication with each other.

In FIG. 6, the fixation device 650 includes an optical system 660 separate from the optical systems 620a and 620b. Generally, the optical system 660 includes a light or image source and one or more lenses for generating and propagating a fixation target through a patient interface 656 and along an optical axis 664 separate from the optical axes 624a and 624b. In certain embodiments, the patient interface 656 includes a lens or other device of the optical system 660, such as an objective lens, for propagating the fixation target along the optical axis 664 to a non-target eye 692 of a patient. In certain embodiments, the optical axis 664 is boresighted with the patient interface 656.

The fixation device 650, the optical system 660, and/or patient interface 656 may be fixedly or adjustably arranged such that the optical axis 664 is aligned, or arranged co-axially, with an optical or visual axis of the non-target eye 692 during use. In certain embodiments, the fixation device 650, and/or components thereof, and the surgical laser device 602, and/or components thereof, are arranged such that the optical axis 664, and the optical axis 624a and/or 624b, are disposed at an interpupillary distance (PD) of a patient. For example, the optical axis 664, and the optical axis 624a and/or 624b are disposed between about 50 mm and about 80 mm from one another, such as between about 60 mm and about 70 mm from one another. In certain embodiments, the distance between the optical axes 664 and 624a and/or 624b, or the distance between the fixation device 650 and the surgical laser device 602, or the distance between the patient interface 656 and the patient interface 606, can be adjusted via the coupling 640. In certain embodiments, the fixation device 650, and/or components thereof, and the surgical laser device 602, and/or components thereof, are arranged such that the optical axis 664, and the optical axis 624a and/or 624b, are parallel or non-parallel to one another.

In certain embodiments, the optical system 660 is in communication with at least one controller 670 of the fixation device 650. Generally, the controller 670 may include a processor and associated memory, and can be utilized to control one or more components of the fixation device 650, such as the optical system 660, in performing one or more functions of the fixation device 650. In certain embodiments, the controller 670 and the controller(s) 630 are one and the same. In other words, the fixation device 650 and the surgical laser device 602 may share a controller.

In certain embodiments, the controller 670 and/or controller 630 are in communication with a user interface of the surgical laser device 602, such as a graphical user interface (GUI) displayed on a screen of the surgical laser device 602, and from which the controller 670 and/or controller 630 may receive user inputs for controlling the fixation device 650. For example, in certain embodiments, the controller 670 and/or controller 630 may receive user inputs from the user interface for controlling a working distance (e.g., focal plane), shape, size, color, brightness, contrast, position, and/or other parameters of the fixation target generated by the fixation device 650. In certain embodiments, the user interface includes a GUI, a touchpad, one or more buttons, a joystick, a foot pedal, or other user input devices. In certain embodiments, the fixation device 650 includes its own user interface for controlling the fixation device, which may include one or more of the user input devices described above.

During use of the surgical laser system 600, the patient may focus the non-target eye 692 on the fixation target generated by the fixation device 650, while light, images, and/or laser light are propagated to and/or from the target eye 690 of the patient by the surgical laser device 602 for performance of one or more functions of the surgical laser device 602.

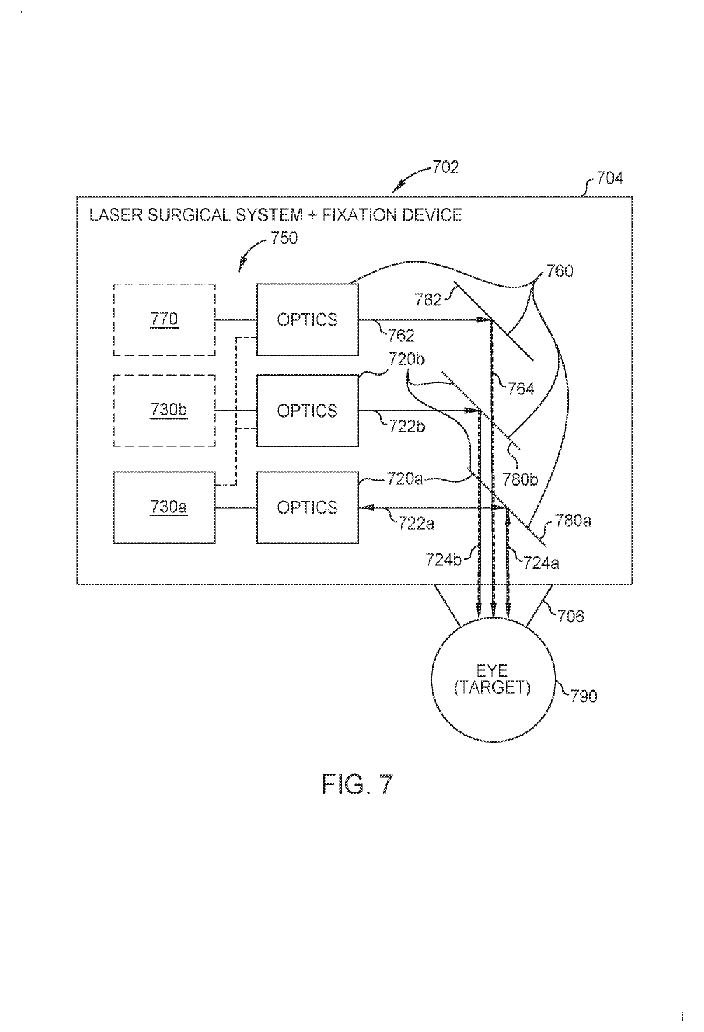

FIG. 7 is a schematic plan view of a surgical laser system 700 with a fixation device 750, according to certain embodiments of the present disclosure. Generally, the surgical laser system 700 can be representative of the surgical laser system 500 described above.

As shown, the surgical laser system 700 includes a surgical laser device 702 that may include, for example, a FLACS device or system. The surgical laser device 702 includes at least one optical system 720a and/or 720b, which may include one or more sensors, detectors, light sources, laser sources, lenses, mirrors, beam splitters, beam combiners, projection systems, reflecting prisms, dispersing devices, filters and thin films, fiber optics, and/or the like for performing one or more functions of the surgical laser device 702. In certain embodiments, the surgical laser device 702 includes at least two optical systems 720a and 720b, wherein each of the at least two optical systems 720a and 720b performs different functions of the surgical laser device 702. The optical system 720a and/or the optical system 720b are at least partially, and in certain embodiments, entirely, disposed within a housing 704 of the surgical laser device 702.

The optical system 720a propagates light for imaging/visualization functions along a bi-directional optical pathway 722a that exits the housing 704 at a patient interface 706 along an optical axis 724a. Imaging and/or visualization performed with the optical system 720a may include near infrared and/or visible imaging techniques. Meanwhile, the optical system 720b propagates laser light for laser treatment functions along an optical pathway 722b that exits the housing 704 at the patient interface 706 along an optical axis 724b. In certain embodiments, the patient interface 706 includes a lens or other device, such as an objective lens, for propagating light along the optical axis 724a and/or optical axis 724b to and/or from a target eye 790 of a patient. Generally, the surgical laser device 702, the optical system 720a, the optical system 720b, and/or the patient interface 706 may be arranged such that the optical axis 724a and/or the optical axis 724b can be aligned, or arranged co-axially, with an optical or visual axis of the target eye 790 during use.

In certain embodiments, the at least two optical systems 720a and 720b can overlap in the surgical laser device 702. For example, in FIG. 7, the optical axis 724a and optical axis 724b are co-axial for at least portions thereof. To facilitate coaxial alignment of the optical axis 724a and the optical axis 724b, the optical system 720a and/or optical system 720b may include one or more optical devices 780 (e.g., illustrated as optical devices 780a and 780b, either or both of which may be referred to as optical device 780) configured to redirect and/or combine light generated and propagated by the optical system 720a and/or optical system 720b. Such optical devices 780 may include mirrors, beam splitters, beam combiners, and/or the like. In FIG. 7, the optical system 720b includes the optical device 780b, which may be a mirror (such as a dichroic mirror), beam splitter, or beam combiner, while both the optical system 720a and the optical system 720b share the optical device 780a, which may be a beam splitter or beam combiner. This arrangement facilitates the overlapping of the optical axis 724a of the optical system 720a and the optical axis 724b of the optical system 720b, which then both pass through the patient interface 706.

The optical system 720a and/or 720b is in communication with at least one controller 730 of the surgical laser device 702. Generally, the controller 730 may include a processor and associated memory, and can be utilized to control one or more components of the surgical laser device 702, such as the optical system 720a and/or 720b, in performing one or more functions of the surgical laser device 702. In certain embodiments, each of the optical system 720a and the optical system 720b are in communication with a corresponding and separate controller 730 (e.g., illustrated as controllers 730a and 730b, respectively, either or both of which may be referred to as controller 730).

The fixation device 750 is integrated with the surgical laser device 702 and is disposed within the housing 704. The fixation device 750 includes an optical system 760 that may include a light or image source and one or more lenses for generating and propagating a fixation target. In the embodiments of FIG. 7, the optical system 760 at least partially overlaps with both the optical system 720a and the optical system 720b. In other words, the optical system 760 and the optical systems 720a and 720b share one or more optical devices (e.g., lenses, mirrors, beam splitters, beam combiners, etc.), and/or form one or more coaxial optical axes. In FIG. 7, the optical system 760 propagates the fixation target along an optical pathway 762 that exits the surgical laser system 702 through the patient interface 706 along an optical axis 764 that is coaxial with the optical axes 724a and 724b. In certain embodiments, the optical axes 724a, 724b, and/or 764 are boresighted with the patient interface 706.

To enable coaxial alignment of the optical axis 764 and the optical axes 724a and 724b, the optical system 760 may include one or more optical devices 782 configured to redirect and/or combine the fixation target generated/propagated by the optical system 760 with light generated/propagated by the optical system 720a and/or the optical system 720b. For example, in certain embodiments, the optical device 782 includes a mirror that redirects the fixation target along the optical axis 764 coaxial with the optical axes 724a and 724b. In certain embodiments, the optical device 782 is shared between the optical system 760 and at least one of the optical system 720a or 720b. In certain embodiments, the optical axis 764 also passes through the optical device 780a and/or 780b, which can be shared between the optical system 760, the optical system 720a, and/or the optical system 720b. Generally, the surgical laser device 702, the optical system 720a, the optical system 720b, the optical system 760, and/or the patient interface 706 may be arranged such that the optical axes 724a and 724b and the optical axis 764 can be aligned, or arranged co-axially, with an optical or visual axis of the target eye 790 during use.

In certain embodiments, the optical system 760 is in communication with at least one controller 770 of the fixation device 750. Generally, the controller 770 may include a processor and associated memory, and can be utilized to control one or more components of the fixation device 750, such as the optical system 760, in performing one or more functions for generating and propagating a fixation target. In certain embodiments, however, the fixation device 750 (and/or optical system 760) shares a controller with the optical systems 720a, 720b, and/or other components of the surgical laser device 702.

In certain embodiments, the controller 770 and/or controller 730 are in communication with a user interface of the surgical laser device 702, such as a graphical user interface (GUI) displayed on a screen of the surgical laser device 702, and from which the controller 770 and/or controller 730 may receive user inputs for controlling the fixation device 750. For example, in certain embodiments, the controller 770 and/or controller 730 may receive user inputs from the user interface for controlling a working distance (e.g., focal plane), shape, size, color, brightness, contrast, position, and/or other parameters of the fixation target generated by the fixation device 750. In certain embodiments, the user interface includes a GUI, a touchpad, one or more buttons, a joystick, a foot pedal, or other user input devices.

During use of the surgical laser device 702, the patient may focus the target eye 790 on the fixation target generated by the optical system 760, while light and/or images are simultaneously propagated to and/or from the target eye 790 by the optical systems 720a and 720b for performance of one or more functions of the surgical laser device 702.

In certain embodiments, additional optical devices such as mirrors (e.g., dichroic mirrors), beam splitters, beam combiners, and the like may be used to split the optical pathway 762 into two optical pathways to direct the fixation target out of the surgical laser device 702 along two optical axes (including optical axis 764). For example, the optical pathway 762 may be split into two pathways, where one pathway passes through the patient interface 706 and the other pathway passes through an additional patient interface (not shown in FIG. 7). This facilitates the propagation of the fixation target to both the target eye 790 and a non-target eye of the patient. In such embodiments, this arrangement may be used to form a three-dimensional fixation target, which may help the patient to maintain better focus as compared to a two-dimensional fixation target propagated to only one eye of the patient.

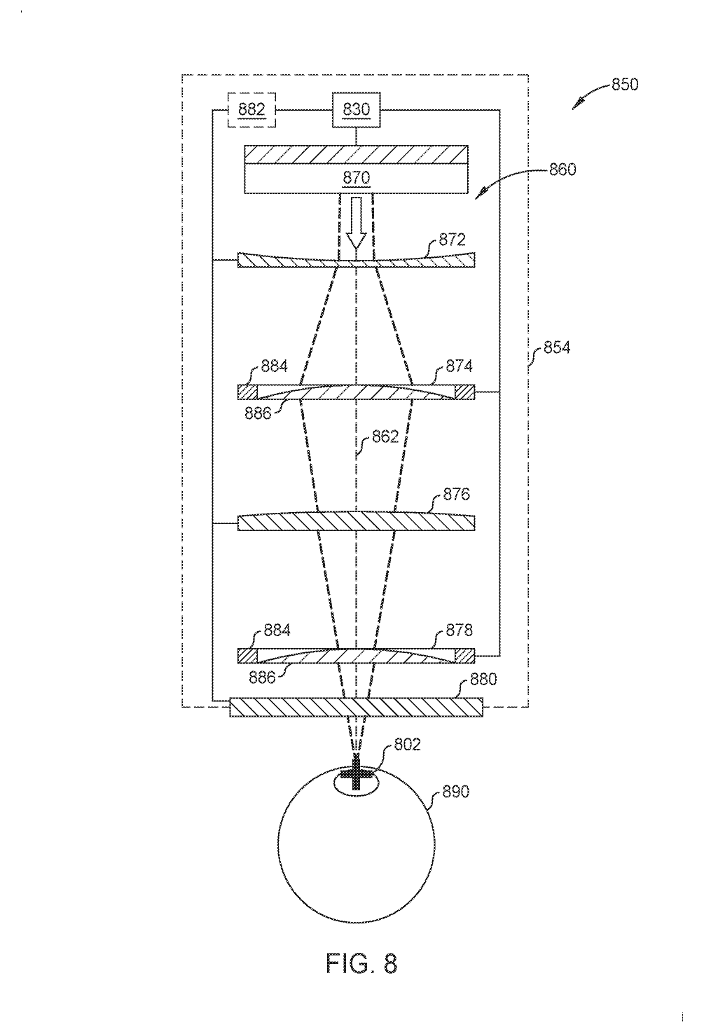

FIG. 8 is a schematic plan view of an exemplary fixation device 850, according to certain embodiments of the present disclosure. The fixation device 850 can be representative of any of the fixation devices of FIGS. 1-7.

As shown, the fixation device 850 includes an optical system 860. The optical system 860 includes a light or image source 870 and a plurality of lenses for generating and propagating a fixation target 802 along an optical pathway 862 toward a patient’s eye 890. In the example of FIG. 8, five lenses 872, 874, 876, 878, and 880 are depicted; however, any suitable number, type, and arrangement of lenses can be utilized.

The light or image source 870 includes a near-to-eye (NTE) display panel configured to generate and project the fixation target 802 to and/or through the lenses 872, 874, 876, 878, and 880 for propagation along the optical pathway 862. Utilization of an NTE display panel enables the fixation device 850 to have a relatively small form factor for easy attachment and/or integration with other devices. Further, the NTE display panel enables the projected fixation target 802 to appear at a distance to the patient, and in certain embodiments, larger than the light or image source 870 and the lenses 872-880 used to create the fixation target 802. It is to be noted that the fixation device 850 is not shown to scale relative to eye 890; instead, the fixation device 850 is enlarged for illustrative purposes. Examples of suitable types of NTE display panels include, but are not limited to, light-emitting diode (LED) display panels, organic light-emitting diode (OLED) display panels, micro-light emitting diode (microLED) display panels, liquid crystal display (LCD) panels, active-matrix liquid crystal display (AMLCD) panels, liquid crystal on silicon (LCOS) display panels, ferroelectric liquid crystal on silicon (fLCOS) display panels, other silicon-based display panels, other microdisplay types, and the like.

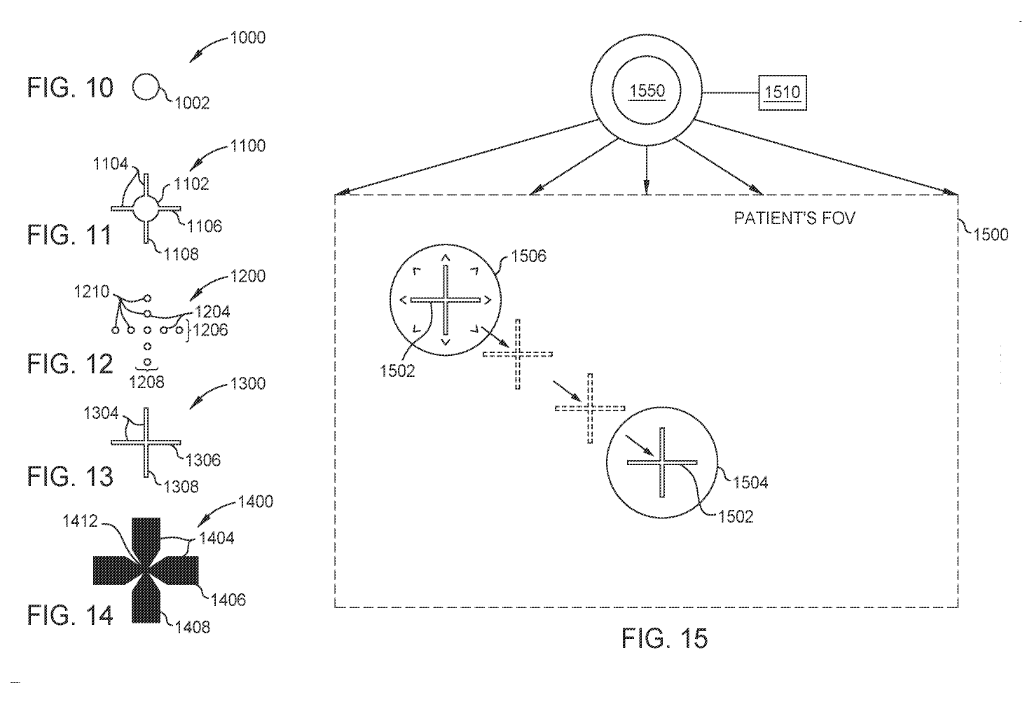

In some embodiments, the light or image source 870 may permit the formation of a cross, circle, or other common shape as the fixation target 802 rather than merely a point. Additionally or alternatively, the light or image source 870 may permit the formation of an image (e.g., a barn, or an apple) as the fixation target 802 upon which the patient may focus. Various examples of such fixation targets 802 are illustrated in FIGS. 10-14.

The light or image source 870 can generate and project the fixation target 802 with any suitable color and wavelength of light based on electrical control signals from at least one controller and/or driver 830, which can be representative of any one or more of the controllers of FIGS. 1-7. For example, in certain embodiments, the light or image source 870 generates the fixation target 802 with white light (400 nm – 700 nm), red light (620 nm – 750 nm), blue light (380 nm – 500 nm), or green light (495 nm – 570 nm). In certain embodiments, the light or image source 870 generates the fixation target 802 with green light at 550 nm, which allows for peak sensitivity of the average human eye 890 when the fixation device 850 is being utilized in an optometric or ophthalmic environment with photopic conditions. In certain embodiments, the light or image source 870 generates the fixation target 802 with red light, which the average human eye 890 is sensitive to under scotopic conditions. In certain embodiments, the light or image source 870 is configured to transition, or shift, between multiple different wavelengths and colors when generating the fixation target 802. In certain embodiments, the color and/or wavelength of the fixation target 802 as generated by the light or image source 870 is selected by a user of the fixation device 850, such as an optometric or ophthalmic technician or medical practitioner, and based on one or more characteristics of a patient. Additionally or alternatively, the wavelength may be selected based on the imaging/visualization and/or treatment lasers of an associated device. For example, if a treatment laser uses UV wavelengths of light, the light or image source 870 may use red light to be further away from the wavelengths used for treatment.

The lenses 872, 874, 876, 878 and 880 relay the fixation target 802 along the optical pathway 862. In the example of FIG. 8, lenses 872, 876, and 880 include traditional optical lenses, which have a set curvature radius and may be formed of glass. One or more of the lenses 872, 876, and 880 (such as all of the lenses 872, 876, and 880) is optional in the fixation device 850. The lenses 872, 876, and 880 may include converging (convex) lenses and/or diverging (concave) lenses. In certain embodiments, a position of one or more of the lenses 872, 876, and 880 along the optical pathway 862 may be adjusted, either manually by the user or by use of an electromechanical motor 882 mechanically coupled to the lenses 872, 876, and/or 880 to adjust a focal plane or size of the fixation target 802. Where an electromechanical motor 882 is used, the electromechanical motor 882 is in electrical communication with, and is controlled by, the at least one controller 830. The electromechanical motor 882 and/or the controller 830 may be of, or in communication with, the fixation device 850.

The lenses 874 and 878, meanwhile, include liquid lenses composed of cells 884 containing an optical-grade liquid material 886 (e.g., water and/or oil) that can change shape in response to electrical input. Accordingly, the lenses 874 and 878 are variable in curvature radius and thus, focal length, based on received electrical control signals. In certain embodiments, electrical control signals are received by the lenses 874 and/or 878 from the at least one controller 830 in electrical communication therewith. Generally, changing the curvature radius of one or both of the lenses 874 and 878 by the transmission of electrical control signals from the controller 830 can be carried out in the order of milliseconds. As a result, a focal length of the lenses 874 and 878, and a focal plane of the fixation target 802, can be adjusted between plus 10 diopters and minus 10 diopters, or more, in about one to two milliseconds, which is generally significantly faster than current techniques for adjusting the focus on traditional optical lenses. As an added benefit, because the liquid lenses 874 and 878 can adjust their focal length while remaining in place, the lenses 874 and 878 can contribute to the relatively small form factor of the fixation device 850. For example, in embodiments where the fixation device 850 includes a few to zero traditional focusing optical lenses, the fixation device 850 will need little to no room to enable translation of the lenses of the optical system 860. Note that although two liquid lenses 874 and 878 are shown, the fixation device 850 may include less than two (e.g., one liquid lens configured to adjust a focus of the fixation device 850 between plus 10 diopters and minus 10 diopters, or more, in about one to two milliseconds) or more than two liquid lenses.

In certain embodiments, a focal plane of the fixation target 802, as generated by the optical system 860, is configured to be disposed at an infinite distance (e.g., the optical system 860 may be configured to focus to infinity). In certain embodiments, the focal plane of the fixation target 802, as generated by the optical system 860, can be adjusted for viewing by a myopic patient, a hyperopic patient, and/or a patient with monovision, while maintaining a focus of the fixation target 802. In certain embodiments, the focal plane of the fixation target 802 can be adjusted to account for refractive error of a patient’s eye, and/or to control the natural accommodation of the patient’s eye. In certain embodiments, the focal plane of the fixation target 802 can be adjusted between plus 10 diopters (or greater) and minus 10 diopters (or less), such as between plus 9 diopters and minus 9 diopters, such as between plus 8 diopters and minus 8 diopters, such as between plus 7 diopters and minus 7 diopters, such as between plus 6 diopters and minus 6 diopters, such as between plus 5 diopters and minus 5 diopters, such as between plus 4 diopters and minus 4 diopters, such as between plus 3 diopters and minus 3 diopters, such as between plus 2 diopters and minus 2 diopters, such as between plus 1 diopter and minus 1 diopter, or a similar range. .

In certain embodiments, the fixation device 850 is a standalone component or device that includes its own housing 854. In such embodiments, one or more of the aforementioned components, including the light or image source 870, the lenses 872, 874, 876, 878, and 880, and the at least one controller 830, are disposed within the housing 854. In certain embodiments, however, the fixation device 850 is integrated into another optometric or ophthalmic diagnostic or surgical device. In such embodiments, the light or image source 870, lenses 872, 874, 876, 878, and 880, and the at least one controller 830 are arranged within the optometric or ophthalmic diagnostic or surgical device, and may be shared or integrated with other optical systems.

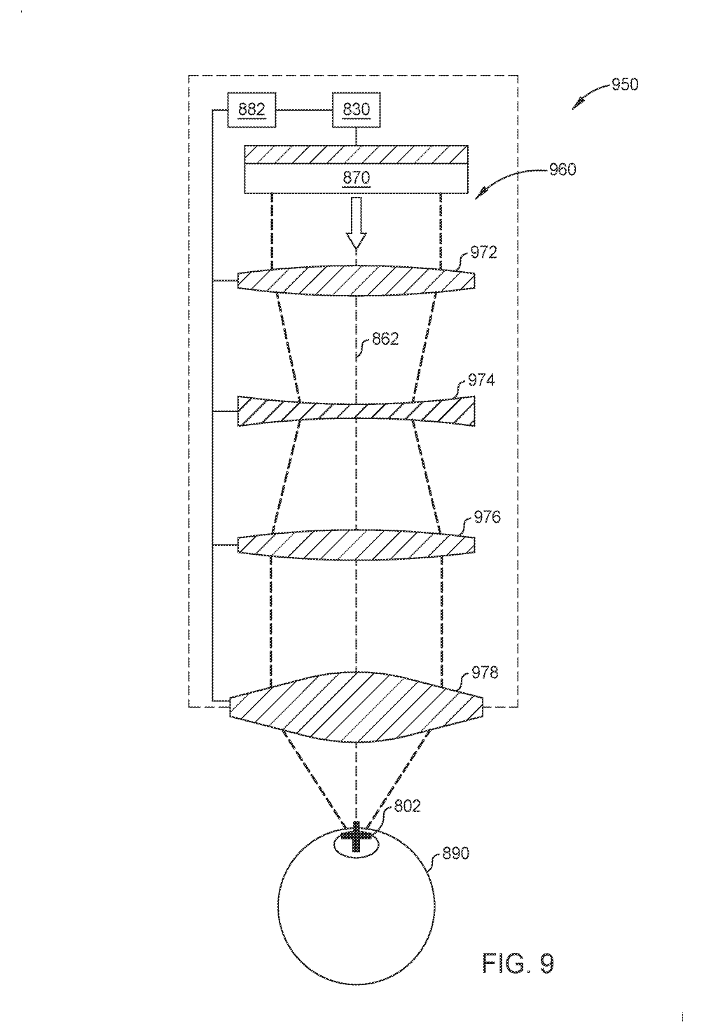

FIG. 9 is a schematic plan view of another exemplary fixation device 950, according to certain embodiments of the present disclosure. Like the fixation device 850, the fixation device 950 can be representative of any of the fixation devices of FIGS. 1-7.

As shown, the fixation device 950 is substantially similar to the fixation device 850, and can generate the fixation target 802 at a wide range of focal planes for patients with a wide variety of ocular/vision conditions. However, unlike the fixation device 850, an optical system 960 of the fixation device 950 includes a plurality of lenses that only includes traditional optical lenses 972, 974, 976, and 978. Accordingly, the focal length of the optical system 960 is adjusted by adjusting a position of one or more of the lenses 972, 974, 976, and 978, either manually by the user or by use of the electromechanical motor 882 mechanically coupled to the lenses 972, 974, 976, and 978.

FIGS. 10-14 depict exemplary fixation targets generated by the fixation devices described herein, according to certain embodiments of the present disclosure. In certain embodiments, a singular fixation device can generate a plurality of different and interchangeable fixation target shapes, as selected by a user and controlled by a corresponding controller of the fixation device.

Generally, the fixation targets described below can be generated in any suitable color of light, including white light (400 nm – 700 nm), red light (620 nm – 750 nm), blue light (380 nm – 500 nm), and green light (495 nm – 570 nm), or combinations thereof. In certain embodiments, the fixation targets described below can be generated with green light at 550 nm, which is a peak sensitivity of the human eye under photopic conditions. In certain embodiments, the fixation targets described below can be generated with red light for scotopic conditions. In certain embodiments, a color of the fixation targets can be transitioned between multiple different wavelengths.

In certain embodiments, the fixation target described below are generated and projected as steady, non-flashing targets. In certain embodiments, the fixation targets described below are generated and projected as flashing, or blinking, targets. Additionally or alternatively, the fixation target may blink or flash in certain conditions and otherwise remain steady.

In FIG. 10, a fixation target 1000 includes a singular circle-like dot 1002.

In FIG. 11, a fixation target 1100 includes a cruciate shape 1104 having intersecting bars 1106 and 1108. A singular circle-like dot 1102 is overlaid and/or intersecting with the cruciate shape 1104 at a center junction of the bars 1106 and 1108. In some embodiments, the fixation target 1100 may transition between the dot 1102 and the cruciate shape 1104, which may create a flashing or blinking appearance.

In FIG. 12, a fixation target 1200 includes a cruciate shape 1204 having intersecting bars 1206 and 1208. Instead of solid lines, each of the bars 1206 and 1208 is formed by linearly-arranged dots 1210. In some embodiments, the fixation target 1200 may alternate which dots 1210 are illuminated, creating an effect of motion towards the center of the cruciate shape 1204, or motion away from the center of the cruciate shape 1204. Additionally or alternatively, such an effect may be created by changing the brightness or color of the dots 1210.

In FIG. 13, a fixation target 1300 includes a cruciate shape 1304 having intersecting bars 1306 and 1308. In FIG. 13, the bars 1306 and 1308 are formed by solid lines. Similarly or comparably to the fixation target 1200 of FIG. 12, the bars 1306 and/or 1308 may have various portions thereof illuminated or colors changed to create an effect of motion towards the center of the cruciate shape 1304, or away from the center of the cruciate shape 1304.

In FIG. 14, a fixation target 1400 includes a cruciate shape 1404 having intersecting bars 1406 and 1408. In FIG. 14, each of the bars 1406 and 1408 increases in thickness, bilaterally, from a center junction 1412 of the bars 1406 to a point along the corresponding bar 1406 or 1408 disposed radially outward form the center junction 1412. Similarly or comparably to the fixation targets 1200 of FIG. 12, the bars 1406 and/or 1408 may have various portions thereof illuminated or colors changed to create an effect of motion towards the center of the cruciate shape 1404, or away from the center of the cruciate shape 1404.

In some examples, cruciate-shaped fixation targets, such as those shown in FIGS. 11-14, may be more effective as compared to traditional fixation spots. For example, patients suffering from certain retinal conditions, such as central scotomas (e.g., blind spots) resulting from macular degeneration, may face difficulties in focusing on, or even visualizing, spot-like patterns. With a cruciate-shaped fixation target, the bars forming the cruciate shape may extend beyond the blind spot of a patient with a central scotoma, thereby making it easier for the patient to locate and focus on the fixation target. Even further, it has been found that single neurons in the visual cortex of the brain can reliably detect straight lines, such as those in the cruciate-shaped fixation targets described above. Thus, the capability to generate a cruciate-shaped fixation target by the fixation devices described herein allows such fixation devices to be more widely effective across the general patient population.

Further, the circular and/or dot-like pattern generated by conventional fixation devices may be difficult to focus on, or even visualize, by patients suffering from certain retinal diseases such as age-related macular degeneration (AMD).

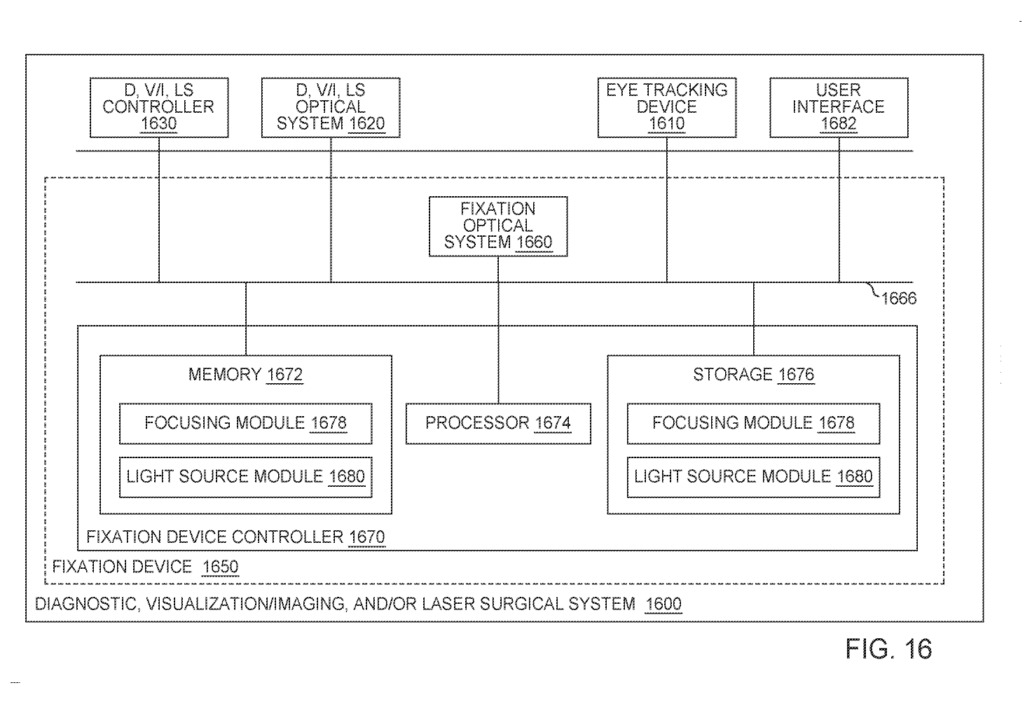

FIG. 15 represents a patient’s field of view (FOV) during performance of eye tracking and visual axis return with an exemplary fixation device 1550, according to certain embodiments of the present disclosure. Generally, the fixation device 1550 can be representative of any of the fixation devices of FIGS. 1- 9 described above.

In some cases, maintaining a patient’s gaze on a centralized fixation target for an extended period of time during an optometric or ophthalmic procedure can be difficult. For example, the patient may become distracted, or tired, and look away from the fixation target, sometimes for prolonged periods of time. Conventionally, the optometric or ophthalmic technician or medical practitioner remains watchful during the procedure to verbally remind/instruct the patient to redirect their gaze onto the fixation target. However, this can cause the technician or medical practitioner’s attention to be diverted from the performance of the actual procedure, which can lead to procedural complications or inefficiencies. Further, the patient may have difficulty re-centering their gaze onto the fixation target after looking away.

In certain embodiments, the fixation device 1550 can be utilized to automatically re-adjust and/or re-center a patient’s gaze to an optimal position, without the need for re-instruction of the patient by the technician or medical practitioner. To facilitate automatic re-adjustment of a patient’s gaze, the device 1550 may be in communication with an eye tracking device 1510. The eye tracking device 1510 may utilize various techniques to track, or determine, eye movements and/or eye positions of a patient. For example, the eye tracking device 1510 may record eye movements using at least one of position sensing detection (PSD) systems, mirror reflection systems, electrooculogram systems, photoelectric and video-based limbus tracking, sclera coils, canthus and corneal bulge tracking, retinal feature tracking, dual Purkinje imaging, dark and bright pupil tracking, pupil and corneal reflection, laser-based pupil and iris tracking, video-based tracking of artificial markers, or pupil center corneal reflections. The eye tracking device 1510 may use bright-pupil or dark-pupil (infrared or near-infrared) techniques. In certain embodiments, the eye tracking device 1510 is integrated with, for example, a diagnostic, visualization/imaging, and/or surgical laser system. In certain embodiments, the eye tracking device 1510 is a separate wearable device.

Referring now to FIG. 15, during use, the fixation device 1550 generates a fixation target 1502 upon which a patient is to focus their gaze during an optometric or ophthalmic procedure. In FIG. 15, the patient’s field of view (FOV) is represented by box 1500. In certain embodiments, the fixation target 1502 is arranged within the patient’s FOV 1500 at a fixation location 1504. In certain embodiments, the fixation location 1504 includes an optimal fixation location where, upon focusing the patient’s gaze onto the fixation location 1504, a least amount of stress is imparted onto the patient’s eye. In certain embodiments, the optimal fixation location 1504 may be substantially central within the patient’s FOV 1500. In certain embodiments, the optimal fixation location 1504 is set by a user via a user interface of, or in communication with, the fixation device 1550.

While the fixation target 1502 is being generated and projected to the patient, the eye tracking device 1510 continuously tracks and determines the position and/or movement of a patient’s eye. In certain embodiments, information relating to the position and/or movement of the patient’s eye is continuously or periodically communicated, either directly or indirectly, between the eye tracking device 1510 and the fixation device 1550, and/or a controller in communication with the fixation device 1550. In certain other embodiments, information relating to the position and/or movement of the patient’s eye is only communicated, either directly or indirectly, to the fixation device 1550, and/or a controller in communication therewith, when the patient’s gaze drifts away from the fixation location 1504.

When the patient’s gaze has drifted away from the fixation target 1502 (and thus, the fixation location 1504), the fixation device 1550 may utilize the information from the eye tracking device 1510 to automatically adjust a position of the fixation target 1502 to be at or near the current location 1506 of the patient’s gaze (via adjustment of the optical system of the fixation device 1550) (e.g., dynamic adjustments). Up until this point, the fixation target 1502 has been generated as a steady, non-flashing illuminated target. However, to secure the patient’s attention once their gaze has drifted off-target, the fixation device 1550 begins flashing, spinning, moving, blinking, or otherwise drawing attention to the fixation target 1502, at or near the current location 1506. Drawing attention to the fixation target 1502 at or near the current location 1506 may elicit saccadic movement of the patient’s eye toward the fixation target 1502.

After a set amount of time at the current location 1506, the position of the fixation target 1502 is slowly moved, or dragged, back to the fixation location 1504 to return the patient’s gaze back onto the fixation location 1504. Generally, the speed of movement of the fixation target 1502 from the current location 1506 to the fixation location 1504 is determined and set to elicit a smooth pursuit movement of the patient’s eye, and not saccadic movement, when returning the patient’s gaze back to the fixation location 1504. In certain embodiments, the fixation target 1502 is flashed during an entirety, or only a portion of, its transition between the current location 1506 and the fixation location 1504. In certain embodiments, flashing of the fixation target 1502 is stopped at the current location 1506 after the set amount of time, and the fixation target 1502 is returned to a steady, non-flashing state prior to its transition between the current location 1506 and the fixation location 1504.

In some embodiments, a change of color may also be used to accentuate the change and/or transition. For example, when moved to the current location 1506, the fixation target 1502 may change from a green color to a flashing red color. While slowly moving or dragging between the current location 1506 and the fixed location 1504, the fixation target 1502 may change to a yellow color; and when arriving at the fixed location 1504, the fixation target 1502 may change to a green color.

Generally, the above technique may be repeated, and/or initiated, each and every time the patient’s gaze has drifted away from the fixation target 1502 and/or the fixation location 1504. Additionally or alternatively, it may be initiated if the patient’s gaze shifts a threshold distance away from the fixed location 1504. Additionally or alternatively, it may be initiated if the patient’s gaze shifts a threshold distance away from the fixed location 1504 for a threshold amount of time.

In some embodiments, using such a device or technique as described and illustrated with reference to FIG. 15, the fixation device 1550 can facilitate dynamically adjusting the fixation target 1502 to help facilitate improved eye fixation and eye focus in a desired location to facilitate treatment of the eye.

FIG. 16 illustrates a schematic diagram of a system 1600, according to embodiments disclosed herein. Generally, the system 1602 is representative of the diagnostic system 100, visualization system 200, biometric and/or visualization system 300, biometric and/or visualization system 400, surgical laser system 500, surgical laser system 600, surgical laser system 700, and/or any other suitable systems or devices for implementation with the fixation devices described herein.

As shown in FIG. 16, the system 1600 includes, or is mechanically coupled to and/or in signal communication with, a fixation device 1650. Generally, the fixation device 1650 is representative of the fixation device 150, fixation device 250, fixation device 350, fixation device 450, fixation device 550, fixation device 650, fixation device 750, fixation device 850, fixation device 950, fixation device 1550, and/or any other fixation devices according to the embodiments described herein.

The fixation device 1650 includes a fixation optical system 1660 for generating and projecting a fixation target, and a fixation device interconnect 1666, which may allow for the communication between components of the fixation device 1650, as well as the communication of the fixation device 1650 with other components of the system 1600. Generally, the fixation optical system 1660 is representative of the optical system 360, optical system 460, optical system 660, optical system 760, optical system 860, and/or optical system 960, as described elsewhere herein.

The fixation device 1650 further includes a fixation device controller 1670 for controlling operations of the fixation device 1650, including operations of the fixation optical system 1660. Generally, the fixation device controller 1670 is representative of the controller 370, controller 430, controller 470, controller 670, controller 730, controller 770, and/or controller 830, as described elsewhere herein.

The fixation device controller 1670 includes a processor 1674, a memory 1672, and a storage 1676. The processor 1674 (e.g., control circuitry) is configured to retrieve and execute programming instructions stored in the memory 1672. Similarly, the processor 1674 may retrieve and store application data residing in the memory 1672. The fixation device interconnect 1666 transmits programming instructions and application data, among the processor 1674, memory 1672, storage 1676, fixation optical system 1660, etc. The processor 1674 may include a single CPU, multiple CPUs, a single CPU having multiple processing cores, and the like. The memory 1672 may be random access memory, and the storage 1676 may be a disk drive. Moreover, the memory 1672 and/or storage 1676 may be any type of a readily available memory, such as random access memory (RAM), read only memory (ROM), floppy disk, hard disk, solid state, flash memory, magnetic memory, or any other form of digital storage, local or remote.

In certain embodiments, the memory 1672 and/or storage 1676 include instructions, which when executed by the processor 1674, cause the fixation optical system 1660 to generate, project, and adjust a focal plane, shape, size, color, and/or other parameters of the fixation target based on inputs from, for example, a user interface 1682 of the system 1600, etc. For example, memory 1672 and/or storage 1676 include a light or image source module 1680 and a focusing module 1678, which may include computer-executable instructions that, when executed by the processor 1674, cause the processor 1674 to control the fixation optical system 1660 in order to generate and project the fixation target based on desired parameters of the fixation target inputted by a user via the user interface 1682. In such an example, the processor 1674 sends output signals via the fixation device interconnect 1666 to the fixation optical system 1660. These output signals allow the processor 1674 to control the operations of the fixation optical system 1660 (e.g., light or image sources, liquid lenses, traditional optic lenses, motors attached to the lenses, etc.) based on the signals from the user interface 1682 for generating a fixation target with desired parameters.

In certain embodiments, the user interface 1682 includes a user interface of the system 1600, or a user interface of the fixation device 1650 itself. The user interface may include a graphical user interface (GUI), a touchpad, one or more buttons, a joystick, a foot pedal, or other user input devices from which the system 1600 and fixation device 1650 may receive user input for controlling, e.g., the fixation device 1650. In certain embodiments, the user interface 1682 may be configured to receive inputs from the user for controlling a working distance (e.g., focal plane), shape, size, color, brightness, contrast, position, and/or other parameters of a fixation target generated by the fixation device 1650.

In the embodiments of FIG. 16, the processor 1674 may include an integrated circuit capable of performing logic functions. In this manner, the processor 1674 is in the form of a standard integrated circuit package with power, input, and output pins. In certain embodiments, the processor 1674 may perform specific control functions targeted to a specific device, such as one or more light or image sources, liquid lenses, etc. In certain embodiments, the processor 1674 is a microprocessor. In certain embodiments, the processor 1674 is not a programmable microprocessor, but instead is a special purpose controller configured to control different components that perform different functions. In some embodiments, the processor 1674, memory 1672, and/or storage 1676 may be implemented as a single standalone chip.

As further shown in FIG. 16, in certain embodiments, the fixation device 1650 is also in signal communication with a controller 1630, an optical system 1620, and/or an eye tracking device 1610 of the system 1600, to coordinate operations therewith. Generally, the controller 1630 can be configured for controlling operations of the system 1600 as related to diagnostic, visualization/imaging, and/or surgical laser functions, which can be carried out by the optical system 1620. Accordingly, the controller 1630 may be representative of the controller 330, 430, 630, and/or 730, and the optical system 1620 may be representative of the optical system 320, optical system 420, optical systems 620a and/or 620b, and/or optical systems 720a and/or 720b, as described elsewhere herein. Similarly, the eye tracking device 1610 may be representative of the eye tracking device 1510, as described elsewhere herein.

The present disclosure may provide improved fixation devices and fixation targets for use during optometric or ophthalmic procedures, including diagnostic and surgical procedures. The described fixation devices and fixation targets may enable more precise fixation by a patient and faster workflow in an optometric or ophthalmic setting, while reducing reliance on the optometric or ophthalmic technicians and/or medical practitioners.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112(f) unless the element is expressly recited using the phrase “means for” or, in the case of a method claim, the element is recited using the phrase “step for.”

While various examples of the subject matter have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘including’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the disclosed subject matter, but instead as merely intended to highlight alternative or additional features that may or may not be utilized in a particular example of the subject matter. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “including as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the disclosed to the specific examples and examples described herein, but rather to also cover all modification and alternatives coming with the true scope and spirit of the subject matter.

What is claimed is:

1. A system, comprising:
an optometric or ophthalmic device;
a fixation device coupled to the optometric or ophthalmic device, the fixation device comprising:
a near-to-eye (NTE) display device, the NTE display device configured to:
generate a fixation target in response to receipt of a first electrical control signal; and
at least one liquid lens, the at least one liquid lens configured to:
propagate the fixation target to an eye of a patient; and
deform in shape in response to receipt of a second electrical control signal, wherein the deformation in shape of the at least one liquid lens changes a focal plane of the fixation target; and
a controller in communication with the fixation device, the controller configured to:
transmit the first electrical control signal to the NTE display device to cause the generation of the fixation target; and
transmit the second electrical control signal to the at least one liquid lens to cause the deformation in shape of the at least one liquid lens.

2. The system of claim 1, wherein the NTE display device comprises at least one of a light-emitting diode (LED) display panel, an organic light-emitting diode (OLED) display panel, a micro-light emitting diode (microLED) display panel, a liquid crystal display (LCD) panel, an active-matrix liquid crystal display (AMLCD) panel, a liquid crystal on silicon (LCOS) display panel, or a ferroelectric liquid crystal on silicon (fLCOS) display panel.

3. The system of claim 1, wherein the NTE display device is configured to generate the fixation target with green light having a wavelength between 495 nm and 570 nm.

4. The system of claim 3, wherein the green light has a wavelength of 550 nm for peak sensitivity of the eye of the patient under photopic conditions.

5. The system of claim 1, wherein the at least one liquid lens is configured to deform in shape within 1 to 2 milliseconds upon receipt of the second electrical control signal.

6. The system of claim 1, wherein the focal plane of the fixation target can be adjusted at least plus and minus 10 diopters to account for refractive error of the eye of the patient.

7. The system of claim 1, wherein the fixation target comprises a cruciate-shaped fixation target.

8. The system of claim 1, further comprising:
an eye tracking device in communication with the controller, wherein the eye tracking device is configured to determine a position or movement of the eye of the patient.

9. The system of claim 8, wherein:
information relating to the determined position or movement of the eye of the patient is communicated between the eye tracking device and the controller, and
in response to receiving the information, the controller is configured to control the fixation device to dynamically adjust a position of the fixation target in a field-of-view (FOV) of the eye of the patient to re-adjust a gaze of the patient.

10. The system of claim 1, wherein the fixation device is adjustably coupled to the optometric or ophthalmic device.

11. The system of claim 1, wherein the fixation device is integrated into the optometric or ophthalmic device.

12. A fixation device, comprising:
a near-to-eye (NTE) display device, the NTE display device configured to:
generate a fixation target in response to receipt of a first electrical control signal;
at least one liquid lens, the at least one liquid lens configured to:
propagate the fixation target to an eye of a patient; and
deform in shape in response to receipt of a second electrical control signal, wherein the deformation in shape of the at least one liquid lens changes a focal plane of the fixation target; and
a controller in communication with the NTE display device and the at least one liquid lens, the controller configured to:
transmit the first electrical control signal to the NTE display device to cause the generation of the fixation target; and
transmit the second electrical control signal to the at least one liquid lens to cause the deformation in shape of the at least one liquid lens.

13. The fixation device of claim 12, wherein the NTE display device comprises at least one of a light-emitting diode (LED) display panel, an organic light-emitting diode (OLED) display panel, a micro-light emitting diode (microLED) display panel, a liquid crystal display (LCD) panel, an active-matrix liquid crystal display (AMLCD) panel, a liquid crystal on silicon (LCOS) display panel, or a ferroelectric liquid crystal on silicon (fLCOS) display panel.

14. The fixation device of claim 12, wherein the NTE display device is configured to generate the fixation target with green light having a wavelength between 495 nm and 570 nm.

15. The fixation device of claim 14, wherein the green light has a wavelength of 550 nm for peak sensitivity of the eye of the patient under photopic conditions.

16. The fixation device of claim 12, wherein the at least one liquid lens is configured to deform in shape within 1 to 2 milliseconds upon receipt of the second electrical control signal.

17. The fixation device of claim 12, wherein the focal plane of the fixation target can be adjusted at least plus and minus 10 diopters to account for refractive error of the eye of the patient.

18. The fixation device of claim 12, wherein the fixation target comprises a cruciate-shaped fixation target.

19. The fixation device of claim 12, wherein the fixation device is configured to adjustably couple to an optometric or ophthalmic device.

20. The fixation device of claim 12, wherein the fixation device is configured to communicate with an eye tracking device configured to determine a position or movement of the eye of the patient.
